(19) United States
(12) Patent Application Publication
(10) Pub. No.: US 2012/0116213 A1
(43) Pub. Date: May 10, 2012

(54) ORIENTATION DETERMINATION OF A MEDICAL DEVICE WITHIN A PATIENT

(76) Inventors: David C. Forster, Los Altos Hills, CA (US); Yenyu Chen, Palo Alto, CA (US)

(21) Appl. No.: 12/942,827

(22) Filed: Nov. 9, 2010

(51) Int. Cl. A61B 6/00; A61B 8/12

(52) U.S. Cl. 600/424

(57) ABSTRACT

Devices and methods for assessing the orientation and shape of vessel lumens and hollow portions of organs are described. The devices and methods are particularly adapted for determining the orientation and shape of a lumen to, for example, perform a valvuloplasty or facilitate the later implantation of a prosthetic heart valve. The devices are typically catheter-based having an expandable member fixed to a distal end of the catheter. Located within the expandable member are materials of different densities wherein the materials of different densities cooperate to indicate the orientation of the device within a lumen. The methods typically comprise deploying the balloon percutaneously to a target location, expanding the balloon, and determining the orientation and shape of a lumen.

### FIELD OF THE INVENTION

The present invention relates generally to medical devices and methods. More particularly, the present invention relates to methods and devices for assessing the orientation, shape, size, topography, compliance, and other aspects of lumens, cardiac valves and surrounding tissue. The devices and methods are particularly adapted for use during minimally invasive surgical interventions, but may also find application during surgical replacement on a stopped heart, less invasive surgical procedures on a beating heart, and other percutaneous procedures.

### BACKGROUND OF THE INVENTION

Minimally invasive surgery provides several advantages over conventional surgical procedures, including reduced recovery time, reduced surgically-induced trauma, and reduced post-surgical pain. Moreover, the expertise of surgeons performing minimally invasive surgery has increased significantly since the introduction of such techniques in the 1980s. As a result, substantial focus has been paid over the past twenty years to devices and methods for facilitating and improving minimally invasive surgical procedures.

One area in which there remains a need for substantial improvement is pre-surgical assessment of treatment locations intended to be subjected to a minimally invasive surgical procedure. For example, when a surgical procedure is to be performed at a treatment location within the body of a patient, it would frequently be beneficial for the surgeon to have prior knowledge of the shape, size, topography, compliance, and other physical properties of the treatment location. This information would be particularly useful in relation to minimally invasive surgical procedures in which prosthetic devices are implanted within a body lumen or within a hollow portion of an organ located within the body of the patient. Such information could then be used to select the size and/or shape of the prosthetic device to more closely match the size, shape, and topography of the treatment location.

A particular portion of the anatomy for which complete and accurate physical assessment would be beneficial are the coronary valves. Diseases and other disorders of heart valves affect the proper flow of blood from the heart. Two categories of heart valve disease are stenosis and incompetence. Stenosis refers to a failure of the valve to open fully, due to stiffened valve tissue. Incompetence refers to valves that cause inefficient blood circulation, permitting backflow of blood in the heart.

Medication may be used to treat some heart valve disorders, but many cases require replacement of the native valve with a prosthetic heart valve. In such cases, a thorough assessment of the shape, size, topography, compliance, and other physical properties of the native valve annulus would be extremely beneficial. Prosthetic heart valves can be used to replace any of the native heart valves (aortic, mitral, tricuspid or pulmonary), although repair or replacement of the aortic or mitral valves is most common because they reside in the left side of the heart where pressures are the greatest.

A conventional heart valve replacement surgery involves accessing the heart in the patent's thoracic cavity through a longitudinal incision in the chest. For example, a median sternotomy requires cutting through the sternum and forcing the two opposing halves of the rib cage to be spread apart, allowing access to the thoracic cavity and heart within. The patient is then placed on cardiopulmonary bypass which involves stopping the heart to permit access to the internal chambers. After the heart has been arrested the aorta is cut open to allow access to the diseased valve for replacement. Such open heart surgery is particularly invasive and involves a lengthy and difficult recovery period.

Less invasive approaches to valve replacement have been proposed. The percutaneous implantation of a prosthetic valve is a preferred procedure because the operation is performed under local anesthesia, does not require cardiopulmonary bypass, and is less traumatic.

### BRIEF SUMMARY OF THE INVENTION

The present invention provides methods and devices for assessing the shape, size, topography, compliance, and other physical properties of a vessel lumen or a hollow portion of another organ located within a patient. The methods and devices may find use in the coronary vasculature, the atrial pendage, the peripheral vasculature, the abdominal vasculature, and in other ducts such as the biliary duct, the fallopian tubes, and similar lumen structures within the body of a patient. The methods and devices may also find use in the heart, lungs, kidneys, or other organs within the body of a patient. Moreover, although particularly adapted for use in vessels and organs found in the human body, the apparatus and methods may also find application in the treatment of animals.

However, the primary use of the methods and devices described herein is in the assessment of the size, shape, topography, compliance, spatial orientation, and other physical properties of the native heart valves and lumens within the human body. Such assessments are useful to facilitate proper orientation, sizing, selection, and implantation of prosthetic heart valves into the native valve space. Proper orientation, selection and sizing ensures that the prosthetic heart valve that is delivered during the implantation procedure will be of a size and shape that fits within the native valve space, including accommodations for any defects or deformities that are detected by the assessment process. Proper orientation, selection and sizing also ensures that the prosthetic valve, once fully expanded, will properly seal against the aortic wall to prevent leakage, and to prevent migration of the prosthetic valve.

The methods and devices described herein are suitable for use in facilitating the orientation, selection and sizing of prosthetic heart valves of all types, independent of the design, implantation mechanism, deployment technique, or any other aspect of the prosthetic valve. In many cases, particularly in the case of a prosthetic valve that is expandable from a delivery state to a deployed state, the assessment of the native valve space is of very great importance. For example, it is important to know the diameter of the native valve space when the valve space has been placed under the expansive load that is produced by the prosthetic valve. If the valve does not fit properly, it may migrate, leak, or resist deployment altogether.

The methods include use of an assessment member that is preferably located at or near the distal end of a catheter or other similar device. The assessment member is introduced to a treatment location within the patient, preferably the native cardiac valve, where the assessment member is activated or otherwise put into use to perform an assessment of one or more physical parameters of the treatment location, to collect the assessment information, and to provide the assessment information to the clinician. Assessment information includes the size (e.g., diameter, circumference, area, volume, etc.) of the valve space, the shape (e.g., round, spherical, irregular, etc.) of the lumen or hollow portion of the organ, the topography (e.g., locations, sizes, and shapes of any irregular features) of the lumen or hollow portion of the organ, the nature of any regular or irregular features (e.g., thrombosis, calcification, healthy tissue, fibrosa) and the spatial orientation (e.g., absolute location relative to a fixed reference point, or directional orientation) of a point or other portion of the treatment location. Access to the treatment location is obtained by any conventional method, such as by general surgical techniques, less invasive surgical techniques, or percutaneously. A preferred method of accessing the treatment location is transluminally, preferably by well-known techniques for accessing the vasculature from a location such as the femoral artery. The catheter is preferably adapted to engage and track over a guidewire that has been previously inserted and routed to the treatment site.

The assessment mechanism includes an expandable member that is attached to the catheter shaft at or near its distal end. The expandable member may comprise an inflatable balloon, a structure containing a plurality of interconnected metallic or polymeric springs or struts, an expandable “wisk”-like structure, or other suitable expandable member. In the case of an inflatable balloon, the expandable member is operatively connected to a source of inflation medium that is accessible at or near the proximal end of the catheter. The expandable member has at least two states, an unexpanded state and an expanded state. The unexpanded state generally corresponds with delivery of the assessment mechanism through the patient's vasculature. The expanded state generally corresponds with the assessment process. The expandable member is adapted to provide assessment information to the user when the expandable member is engaged with a treatment location within the body of a patient.

Turning to several exemplary devices and methods, in one aspect of the invention, a catheter-based system includes a transluminal imaging device contained partially or entirely within an expandable structure attached at or near the distal end of the catheter. The imaging device may comprise any suitable acoustic or other device used for imaging processes, such as intravascular ultrasonic imaging processes. In the preferred embodiment, the imaging device is an ultrasonic imaging probe that is configured to transmit and receive ultrasonic signals at a desired frequency or at a plurality of desired frequencies. The received signals are then used to calculate measurement information, which measurement information is then captured for later use or displayed to the clinician through a suitable display. Further, the received signals in combination with materials within the expandable member may be used to determine the orientation of the expandable member within the patient.

In the preferred embodiments, the expandable member is a balloon member. The balloon member is connected to an inflation lumen that runs between the proximal and distal ends of the catheter, and that is selectively attached to a source of inflation medium at or near the proximal end of the catheter. The balloon member is thereby selectively expandable while the imaging device is located either partially or entirely within the interior of the balloon. The imaging device is adapted to be advanced, retracted, and rotated within the balloon, thereby providing for imaging in a plurality of planes and providing the ability to produce three-dimensional images of the treatment site.

In a preferred embodiment, the expandable member may have at least two materials having different densities. The two materials cooperate to indicate the orientation of the expandable member with respect to the relative orientation of the patient. One of the materials may be a radiopaque material which in cooperation with fluoroscopic visualization or other suitable means may determine the orientation of the expandable member.

In a further preferred embodiment, a longitudinal medical device, such as a catheter or a wire, for inserting into a lumen or hollow portion of an organ is fitted with a gravitational orientation means to indicate the orientation of the medical device within the lumen or hollow portion of the organ.

In optional embodiments, the expandable member is filled with a medium that enhances the imaging process. For example, the medium may comprise a material that increases the transmission capabilities of the ultrasonic waves, or that reduces the amount of scattering of the ultrasonic waves that would otherwise occur without use of the imaging-enhancing medium. In still other optional embodiments, the expandable structure contains (e.g., has embedded or formed within) or is formed of a material that enhances the imaging process. In still other embodiments, the expandable member includes a layer of or is coated with a material that enhances the imaging process.

In use, the transluminal imaging device is first introduced to the target location within the patient, such as the native valve annulus. In the preferred embodiment, this is achieved by introducing the catheter through the patient's vasculature to the target location. Typically, the catheter tracks over a guidewire that has been previously installed in any suitable manner. The imaging device may be provided with a radiopaque or other suitable marker at or near its distal end in order to facilitate delivery of the imaging device to the target location by fluoroscopic visualization or other suitable means. Once the imaging device is properly located at the target location, the expandable structure is expanded by introducing an expansion medium through the catheter lumen. The expandable structure expands such that it engages and applies pressure to the internal walls of the target location, such as the valve annulus. The expandable structure also takes on the shape of the internal surface of the target location, including all contours or other topography. Once the expandable structure has been sufficiently expanded, the imaging device is activated. Where appropriate, the imaging device is advanced, retracted, and/or rotated to provide sufficient movement to allow a suitable image of the target location to be created, or to collect a desired amount of measurement information. The measurement information collected and/or the images created by the imaging device are then transmitted to a suitable user interface, where they are displayed to the clinician.

In use, the expandable member is first introduced to the target location within the patient. In the preferred embodiment, this is achieved by introducing the catheter through the patient's vasculature to the target location. The catheter tracks over a guidewire that has been previously installed in any suitable manner. The expandable member carried on the catheter may be provided with a radiopaque or other suitable marker at or near its distal end in order to facilitate delivery of the physical assessment member to the target location by fluoroscopic visualization or other suitable means. Once the expandable member is properly located at the target location, the expandable member is expanded by introducing an expansion medium through the catheter lumen. The expandable member expands to a predetermined size such that the expandable member is able to engage the lumen or hollow portion of the organ, thereby providing an indicator of the shape and orientation of the lumen or hollow portion of the organ. In this way, the clinician is able to obtain precise measurements of the shape and orientation of the lumen or hollow portion of the organ at the target location. In a further preferred embodiment, the expandable member may be expanded to a size greater than the lumen or hollow portion of the organs to provide additional assessment information.

In a further aspect of the present invention, a valvuloplasty procedure is performed in association with the assessment of the native cardiac valve. In a first embodiment, the expandable member also functions as a valvuloplasty balloon. The expandable member is placed within the cardiac valve space, where it is expanded. Expansion of the expandable member causes the native valve to increase in size and forces the valve, which is typically in a diseased state in which it is stiff and decreased in diameter, to open more broadly. The valvuloplasty procedure may therefore be performed prior to the deployment of a prosthetic valve, but during a single interventional procedure. In a further preferred embodiment, the expandable member after performing valvuloplasty may be expanded beyond the shape and size of the native cardiac valve to distort the native cardiac valve and perform an assessment function.

The measurement and diagnostic processes performed by any of the foregoing devices and methods may be used to facilitate any suitable medical diagnosis, treatment, or other therapeutic processes. One particular treatment that is facilitated by the foregoing devices and methods is the repair and/or replacement of coronary valves, particularly aortic valve replacement using a prosthetic valve.

Other aspects, features, and functions of the inventions described herein will become apparent by reference to the drawings and the detailed description of the preferred embodiments set forth below.

### DESCRIPTION OF THE DRAWINGS

FIG. 1 is a perspective view of a catheter in accordance with several of the embodiments of the present invention.

FIG. 2A is a cross-sectional view of an imaging device in accordance with the present invention.

FIG. 2B is a cross-sectional view of the imaging device of FIG. 2A, showing an expandable member in its expanded state.

FIG. 3 is an illustration of an image of a diseased lumen.

FIG. 4 is a cross-sectional view of a first exemplary embodiment of an expandable member in its expanded state having two materials of differing density and an imaging device.

FIG. 5 is a cross-sectional view of a second exemplary embodiment of an expandable member in its expanded state having two materials of differing density and an imaging device.

FIG. 6 is a cross-sectional view of a third exemplary embodiment of an expandable member in its expanded state having two materials of differing density and an imaging device.

FIG. 7 is a cross-sectional view of a fourth exemplary embodiment of an expandable member in its expanded state having two materials of differing density and no imaging device.

FIG. 8A is a cross-sectional view of a fifth exemplary embodiment of an expandable member in its expanded state having two materials of differing density and no imaging device and FIG. 8B is a cross-sectional view of FIG. 8A in the direction of arrows 8B-8B.

FIG. 9 is a cross-sectional view of a sixth exemplary embodiment of an expandable member in its expanded state having two materials of differing density and an imaging device.

FIG. 10 is a cross-sectional view of a seventh exemplary embodiment of an expandable member in its expanded state having two materials of differing density and an imaging device.

FIG. 11 is an exemplary embodiment of a longitudinal medical device having a gravitational orientation means.

### DETAILED DESCRIPTION OF THE PREFERRED EMBODIMENTS

The present invention is directed to methods and devices for assessing the orientation, shape, size, topography, contours, and other aspects of anatomical vessels and organs using minimally invasive surgical techniques. As summarized above, the devices are typically catheter-based devices. Such devices are suitable for use during less invasive and minimally invasive surgical procedures. However, it should be understood that the devices and methods described herein are also suitable for use during surgical procedures that are more invasive than the preferred minimally invasive techniques described herein.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which these inventions belong. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present inventions.

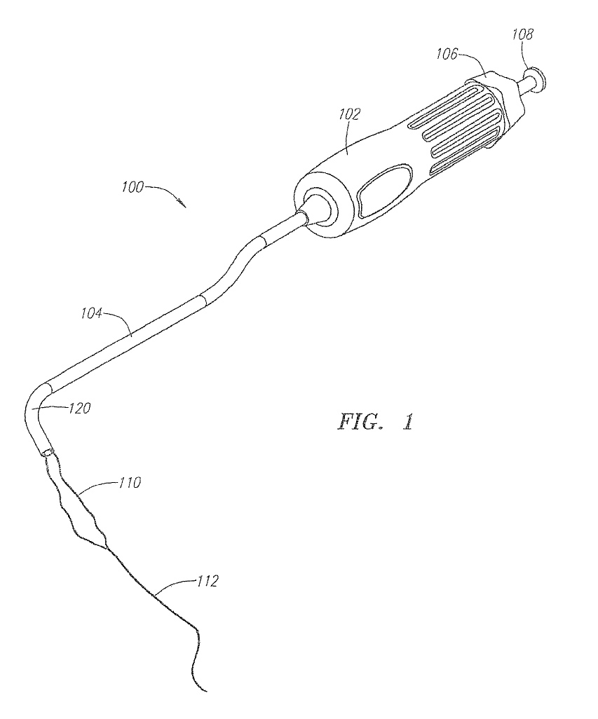

Turning to the drawings, FIG. 1 shows a catheter 100 suitable for use with each of the assessment mechanisms described herein. The catheter 100 includes a handle 102 attached to the proximal end of an elongated catheter shaft 104. The size and shape of the handle 102 may vary, as may the features and functionality provided by the handle 102. In the illustrated embodiment, the handle 102 includes a knob 106 rotatably attached to the proximal end of the handle 102. The knob 106 may be rotated to control the movement and/or function of one or more components associated with the catheter 100, such as for retraction of one or more catheter shafts or sheaths, or manipulation of an expandable member or other component carried at or near the distal end of the catheter shaft 104. Alternative structures may be substituted for the knob 106, such as one or more sliders, ratchet mechanisms, or other suitable control mechanisms known to those skilled in the art.

An inflation port 108 is located near the proximal end of the handle 102. The inflation port 108 is operatively connected to at least one inflation lumen that extends through the catheter shaft 104 to an expandable member 110 located near the distal end of the catheter shaft 104. The inflation port 108 is of any suitable type known to those skilled in the art for engaging an appropriate mechanism for providing an inflation medium to inflate the expandable member 110. For example, a suitable inflation mechanism is an Indeflator™ inflation device, manufactured by Guidant Corporation.

The catheter 100 is adapted to track a guidewire 112 that has been previously implanted into a patient and routed to an appropriate treatment location. A guidewire lumen extends through at least the distal portion of the catheter shaft 104, thereby providing the catheter 100 with the ability to track the guidewire 112 to the treatment location. The catheter 100 may be provided with an over-the-wire construction, in which case the guidewire lumen extends through the entire length of the device. Alternatively, the catheter 100 may be provided with a rapid-exchange feature, in which case the guidewire lumen exits the catheter shaft 104 through an exit port at a point nearer to the distal end of the catheter shaft 104 than the proximal end thereof.

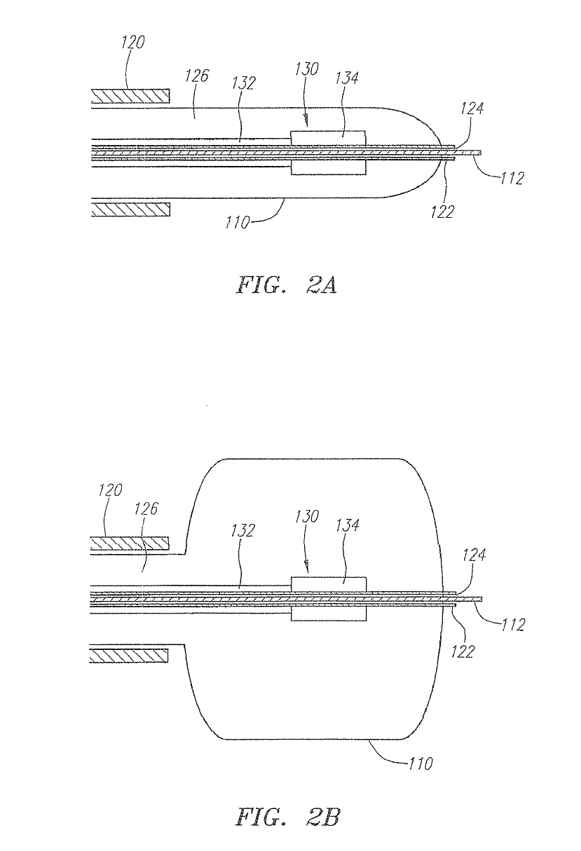

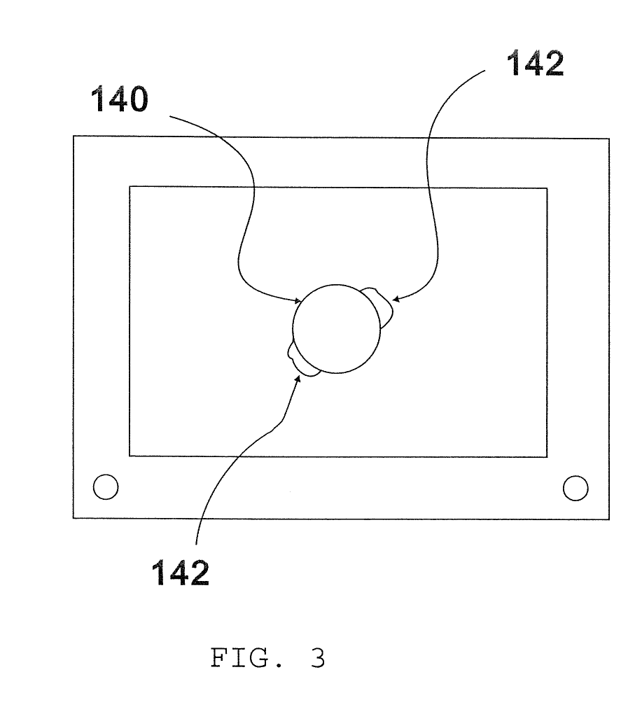

Turning next to FIGS. 2A-B, an assessment mechanism is shown and described. The assessment mechanism is located at the distal end of a catheter 100, such as that illustrated in FIG. 1 and described above. The assessment mechanism shown in FIGS. 2A-B includes an imaging device that is used to provide two-dimensional or three-dimensional images of a vessel lumen or the hollow portion of an organ within the body of a patient, as described below.

The assessment mechanism includes the outer sheath 120 of the catheter shaft 104, which surrounds the expandable member 110. In the preferred embodiment, the expandable member 110 is an inflatable balloon. The expandable member 110 is attached at its distal end to a guidewire shaft 122, which defines a guidewire lumen 124 therethrough. The guidewire 112 extends through the guidewire lumen 124.

An imaging member 130 is contained within the expandable member 110. The imaging member 130 is supported by a shaft 132 that extends proximally to the handle 102, where it is independently controlled by the user. The imaging member shaft 132 is coaxial with and surrounds the guidewire shaft 124, but is preferably movable (e.g., by sliding) independently of the guidewire shaft 124. At the distal end of the imaging member shaft 132 is the imaging head 134. The imaging head 134 may be any mechanism suitable for transmitting and receiving ultrasonic waves. A typical imaging head 134 is an ultrasonic imaging probe for ultrasound imaging. Alternatively, the imaging member 130 may be an optical fiber in conjunction with optical coherence tomography for optical imaging or an acoustic device for transesophageal echo. The expandable member 110 is subject to expansion when a suitable expansion medium is injected into the expandable member through the inflation lumen 126. The inflation lumen 126, in turn, is connected to the inflation port 108 associated with the handle 102. FIG. 2A illustrates the expandable member 110 in its unexpanded (contracted) state, while FIG. 2B illustrates the expandable member 110 in its expanded state, such as after a suitable inflation medium is injected through the inflation port 108 and inflation lumen 126 into the expandable member 110.

To use the assessment mechanism illustrated in FIGS. 2A-B, the distal portion of the catheter is delivered to a treatment location within the body of a patient over the previously deployed guidewire 112. In a particularly preferred embodiment, the treatment location is the aortic heart valve, and the guidewire 112 is deployed through the patient's vasculature from an entry point in the femoral artery using, for example, the Seldinger technique. Deployment of the assessment mechanism is preferably monitored using fluoroscopy or other suitable visualization mechanism. Upon encountering the treatment location, the expandable member 110 is expanded by inflating the balloon with a suitable inflation medium through the inflation port 108 and the inflation lumen 126. The expandable member 110 engages the internal surfaces of the treatment location, such as the annular root of the aortic heart valve. Once the expandable member 110 is expanded, the imaging head 134 is activated and the imaging process is initiated. The imaging head 134 is preferably advanced, retracted, and rotated within the expandable member 110 as needed to obtain images in a variety of planes to yield a 360° three-dimensional image, or any desired portion thereof. Once the imaging process is completed, the expandable member 110 is deflated, and the assessment mechanism may be retracted within the catheter shaft 104. The catheter 100 is then removed from the patient.

Optionally, the inflation medium used to expand the expandable member 110 may comprise a material that enhances the ability of the imaging head 134 to generate images. For example, the inflation medium may facilitate enhanced acoustic transmission, reception, or it may reduce the incidence of scattering of the assessment signal. Such suitable inflation media may include a liquid or a gas and more specifically may include, for example, the following: acoustic gel, dielectric fluid, saline, and the like. These effects may be enhanced further by provision of a material or coating on the surface of the expandable member 110 that optimizes the imaging process. Such suitable materials and/or coatings include relatively dense materials such as metal, ceramic, high density polymers, and the like.

An exemplary embodiment of the present invention pertains to determining the relative position of a device or image with respect to a patient's position. After a medical device has been placed in a patient, the medical device will be in an unknown orientation with respect to the orientation of the patient. For example, referring to FIG. 3, there is shown an illustration of a patient's lumen 140. The patient's lumen is in a diseased condition and contains several areas of plaque 142. However, the orientation of the plaque with respect to the patient is not known. Knowing the orientation of the anatomical features in a patient, the orientation of disease and the orientation of a medical device is critical information.

Often, the orientation of the distal end of the expandable member and the proximal end that the physician uses are not synchronized and other means may need to be used to determine orientation. That is, the handle 102 of the catheter 100 that the physician uses may have a different orientation than the distal end of the expandable member due to the twisting of the catheter as it passes through the patient's body. Accordingly, the physician may not know the orientation of the features of a patient's lumen, such as plaque, with respect to the orientation of the patient.

The present invention in exemplary embodiments uses materials of two different densities. The material of higher density may be locationally positioned in a known way due to the force of gravity. In some of the exemplary embodiments, there is only one material of such a nature that the appearance of the material indicates its gravitational orientation. As described in the exemplary embodiments, the materials for use herein may be chosen from a wide variety of solid, liquid, and gaseous materials.

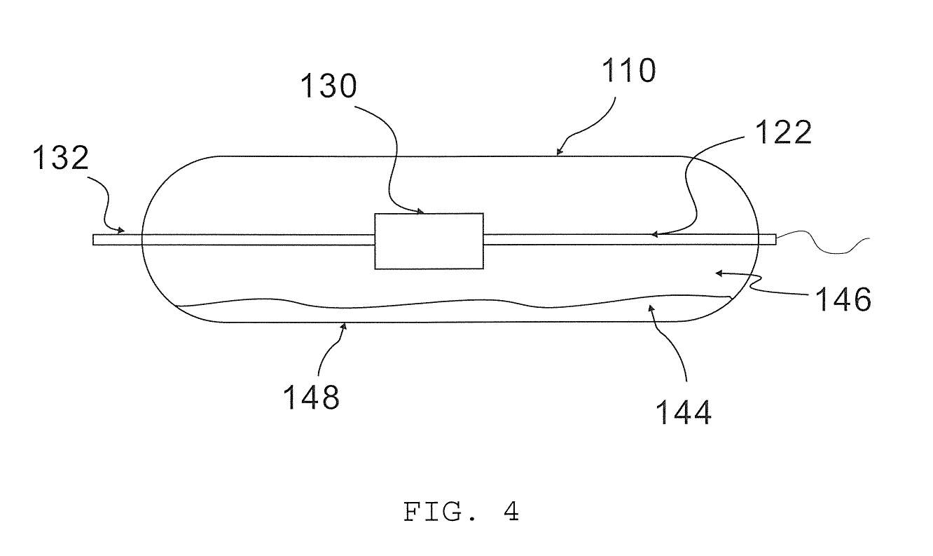

Referring to FIG. 4, an exemplary embodiment is illustrated. Within expandable member 110, there is a first material 144 having a first density and second material 146 having a second density. These materials are non-toxic fluids and/or gases. The density of the first material 144 is greater than the density of the second material 146 so that first material 144 will sink to the bottom 148 of expandable member 110. Then, when the expandable member 110, first material 144 and second material 146 are imaged, such as by imaging device 130, the first material 144 will be in a known position due to the force of gravity. Then, the orientation of any anatomical feature in a patient can be determined by using the orientation of the first material 144 as a reference point. In this exemplary embodiment, first material 144 and second material 146 are both liquids. It is understood that the first and second materials 144 and 146 may both be gases or a liquid and a gas as long as the first material 144 has a greater density than the second material 146. Typical high density materials may be, but are not limited to, water, oil, saline, contrast fluids, and heavy gases. Typical low density materials are carbon dioxide, air, oxygen, a light gas relative to the aforementioned heavier gas, and water. It is understood that the first and second materials may be used as the inflation medium to expand expandable member 110.

The imaging device 130 may be any of the imaging devices, including an ultrasound imaging device, discussed previously.

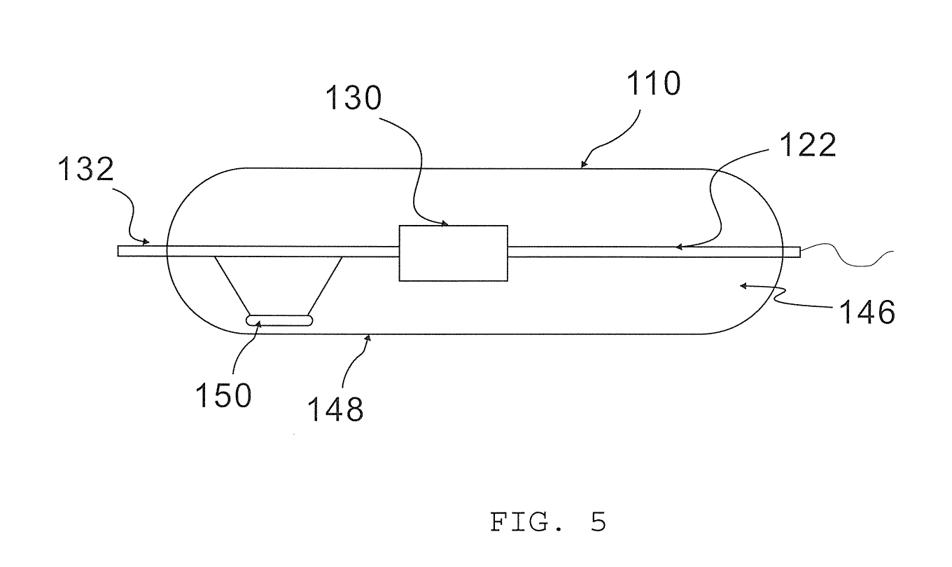

An alternative exemplary embodiment is illustrated in FIG. 5. In this embodiment, the second material is generally in the shape of a cylinder shown as rod 150. Rod 150 is extended from imaging device shaft 132. Alternatively, rod 150 may be free to move about the inside of the expandable member 110 without any attachment to the imaging member shaft 132. In this exemplary embodiment, rod 150 is the first material and has a higher density than second material 146. Rod 150 may be made from a material such as, but not limited to, lead, iron, copper, stannum, tungsten, platinum, gold, silver, and tantalum. Second material 146 may be a liquid or a gas such as any of the materials listed above with respect to the FIG. 4 embodiment. Due to its higher density, rod 150 will sink to the bottom 148 of expandable member 110.

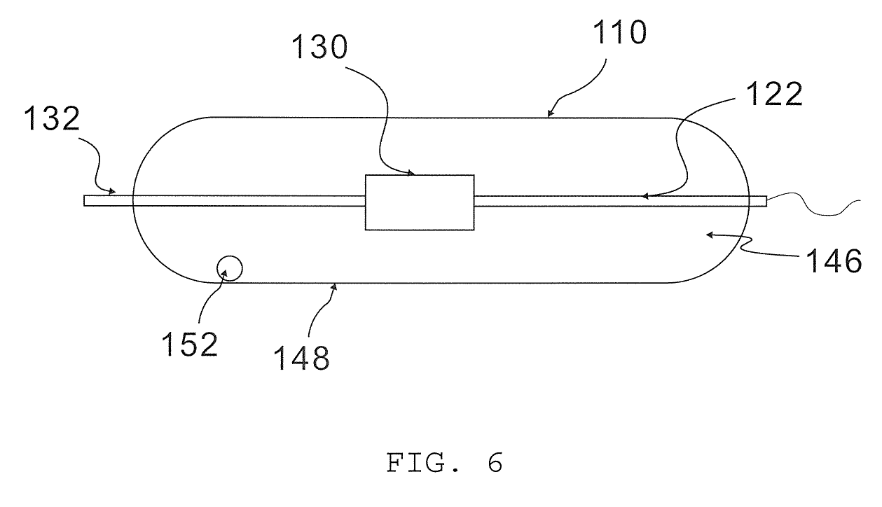

A further alternative exemplary embodiment is illustrated in FIG. 6 wherein the first material is generally in the shape of a sphere shown as ball 152. The ball 152 has a higher density than first material 146. The ball 152 may be made from any of the materials listed above with respect to rod 150. Second material 146 may be a liquid or a gas such as any of the materials listed above with respect to the FIG. 4 embodiment. Due to its higher density, ball 152 will sink to the bottom 148 of expandable member 110.

Another alternative exemplary embodiment is illustrated in FIG. 7 wherein the first material is a ball 154. The ball 154 has a higher density than first material 146. In this embodiment, there may not be an imaging device or imaging device shaft within the expandable member 110 as shown in the previous exemplary embodiments. In the exemplary embodiment illustrated in FIG. 7, ball 154 is made from a radiopaque material such as those listed above for rod 150. Second material 146 may be a liquid or a gas such as any of the materials listed above with respect to the FIG. 4 embodiment. Due to its higher density, ball 154 will sink to the bottom 148 of expandable member 110. Since ball 154 is made from a radiopaque material, its orientation can be readily determined by fluoroscopic visualization or other similar means from outside the patient's body. It is within the scope of the invention that rod 150, ball 152, and ball 154 need not be made from a radiopaque material and its orientation can be determined by a means such as ultrasound imaging by an ultrasonic imaging probe from outside the body of the patient.

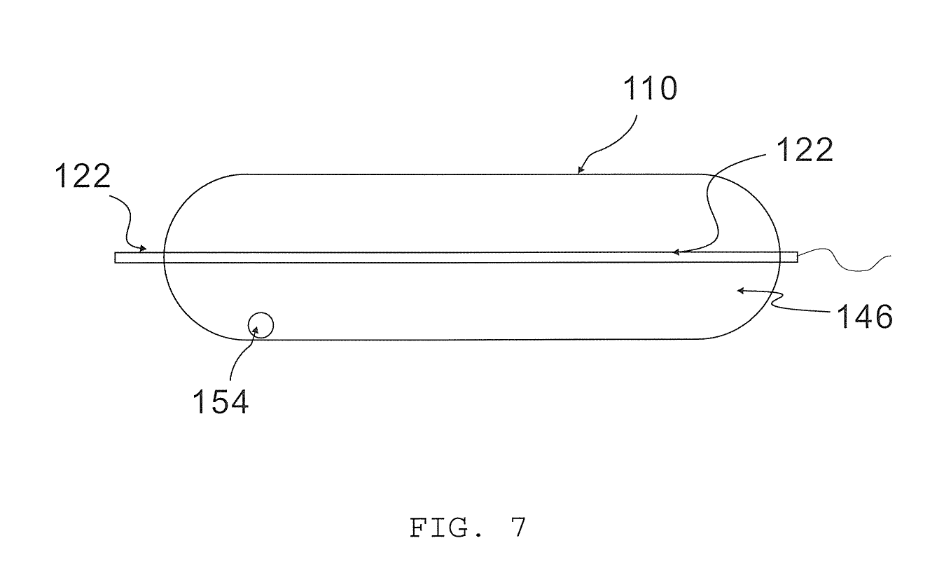

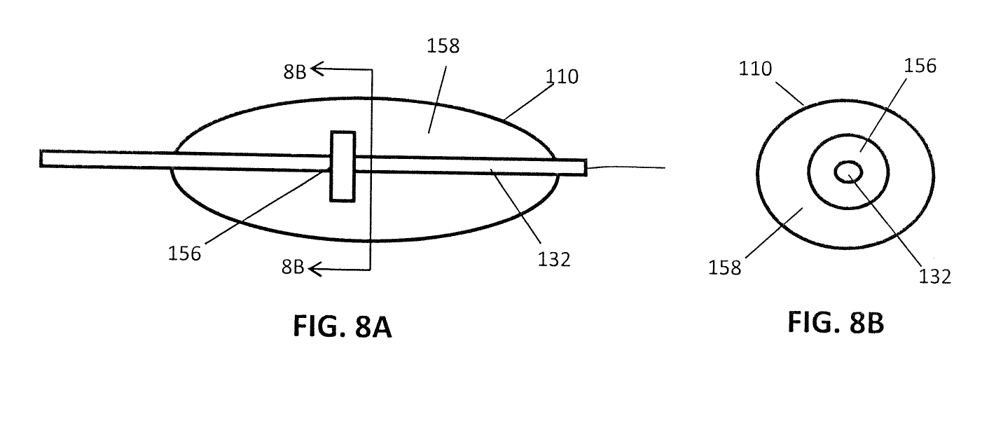

Referring to FIGS. 8A and 8B, there is an exemplary embodiment in which a collar 156 is placed around imaging member shaft 132. Collar 156 contains gas bubbles, for example air, oxygen, nitrogen, or other nontoxic gas. Expandable member 110 also contains a fluid medium 158 for expanding the expandable medium. Since the gas bubbles are wholly contained within collar 156, the relative densities of fluid medium 158 and the gas bubbles is not relevant. In this exemplary embodiment, any imaging means would be present outside the body of the patient. During imaging, the gas bubbles would naturally float up and opposite to the force of gravity, thereby indicating the orientation of the expandable member 110 with respect to the body of the patient.

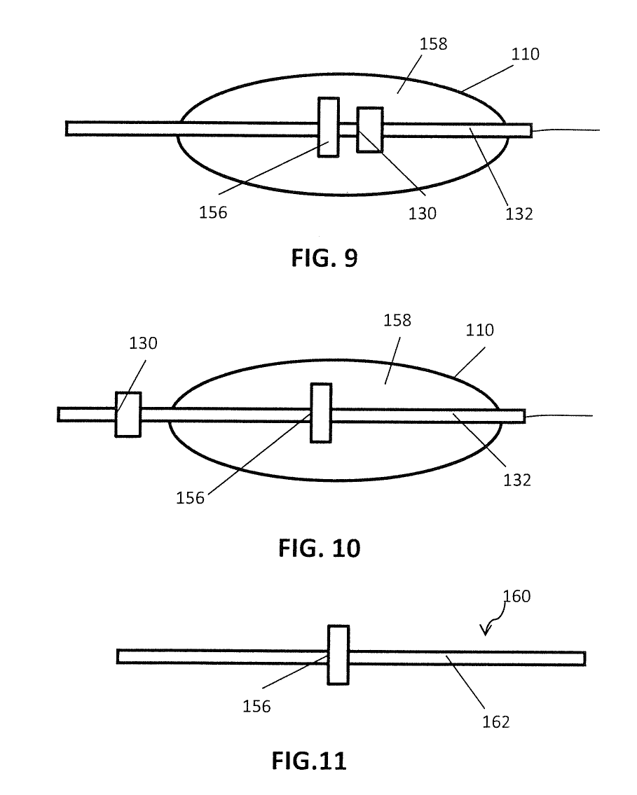

A further exemplary embodiment is illustrated in FIG. 9 in which there is an imaging member 130 within the expandable member 110 for imaging the gas bubbles in collar 156.

The exemplary embodiment shown in FIG. 10 is similar to the embodiment shown in FIG. 9 except now the imaging member 130 is located outside of the expandable member 110. While not shown in FIG. 10, the collar 156 having the gas bubbles can be located outside of the expandable member 110 while the imaging member 130 is located within the expandable member 110.

In addition to the use of the expandable member 110 having first material of a first density and second material of a second density to determine the orientation of an anatomical feature, there may also be performed an assessment procedure to determine at least one physical property of a lumen, such as the shape, size, topography, compliance or other physical property.

Referring now to FIG. 11, a last exemplary embodiment is illustrated. A medical device 160 is shown. Such a medical device 160 may include, for example, a longitudinal member 162 such as a catheter or wire that may be inserted into a lumen or hollow portion of an organ of a patient. Located on the longitudinal member 162 may be a collar 156 having gas bubbles as described above. Such a collar 156 would be advantageous in determining the orientation of the longitudinal member 162 with respect to the orientation of the patient. External imaging means may be used to determine the orientation of the longitudinal member 162.

All of the embodiments of collar 156 discussed above have first and second materials of different densities, specifically, a liquid and a gas. It is within the scope of the invention to use two liquids of different density in such a collar 156. It is also understood that collar 156 may be shaped as desired to surround or partially surround and attach to any medical device in three dimensions to determine the orientation of the medical device within the spirit of the claimed invention.

The preferred embodiments of the inventions that are the subject of this application are described above in detail for the purpose of setting forth a complete disclosure and for the sake of explanation and clarity. Those skilled in the art will envision other modifications within the scope and spirit of the present disclosure. Such alternatives, additions, modifications, and improvements may be made without departing from the scope of the present inventions, which is defined by the claims.

What is claimed is:

1. A medical device comprising:
a catheter shaft having a proximal end and a distal end;
an expandable member carried at or near the distal end of the catheter,
a first material having a first density within the expandable member; and
a second material having a second density within the expandable member, wherein the first density is different from the second density, the first and second materials cooperating to indicate the orientation of the device within a lumen.

2. The medical device of claim 1 further comprising a medium for expanding the expandable member.

3. The medical device of claim 2 wherein the expanding medium is a liquid or a gas.

4. The medical device of claim 2 wherein the expanding medium is selected from the group consisting of saline, acoustic gel, dielectric fluid, blood, gas and contrast medium.

5. The medical device of claim 1 wherein the expandable member is a balloon.

6. The medical device of claim 2 wherein the first material and the expanding medium are the same materials.

7. The medical device of claim 1 wherein the second material is generally in the shape of a sphere.

8. The medical device of claim 1 wherein the second material is generally in the shape of a cylinder.

9. The medical device of claim 1 further including an imaging device to show the orientation of the device within the lumen.

10. The medical device of claim 9 wherein the imaging device is within the expandable member.

11. The medical device of claim 9 wherein the imaging device is outside of the expandable member.

12. The medical device of claim 9 wherein the imaging device is an ultrasonic imaging head for ultrasound imaging.

13. The medical device of claim 9 wherein the imaging device is an optical fiber for optical coherent imaging.

14. A medical device comprising:
a catheter shaft having a proximal end and a distal end;
an expandable member carried at or near the distal end of the catheter,
a collar located on the catheter shaft, the collar being a hollow member containing gas bubbles to indicate the orientation of the device within a lumen.

15. The medical device of claim 14 wherein the collar is located within the expandable member.

16. The medical device of claim 14 wherein the collar is located outside of the expandable member.

17. The medical device of claim 14 further comprising an imaging device to show the orientation of the device within the lumen.

18. The medical device of claim 14 wherein the imaging device is within the expandable member.

19. The medical device of claim 14 wherein the imaging device is outside of the expandable member.

20. The medical device of claim 14 wherein the imaging device is an ultrasonic imaging head for ultrasound imaging.

21. The medical device of claim 14 wherein the imaging device is an optical fiber for optical coherent imaging.

22. A method for assessing the shape and orientation of a lumen comprising:
deploying an expandable member to the location of a lumen within the body of a patient, the expandable member being attached to a catheter at or near a distal end thereof, the expandable member having a first material having a first density within the expandable member, and a second material having a second density within the expandable member, wherein the first density is different from the second density;
expanding the expandable member to engage at least a portion of the lumen; and
imaging by an imaging device the first and second materials to show the arrangement of the first and second materials, the arrangement of the first and second materials indicating relative orientation of the expandable device with respect to the patient.

23. The method of claim 22 further comprising a medium for expanding the expandable member.

24. The method of claim 23 wherein the medium is a liquid or a gas.

25. The method of claim 23 wherein the expanding medium is selected from the group consisting of saline, acoustic gel, dielectric fluid, blood, gas and contrast medium.

26. The method of claim 22 wherein the expandable member is a balloon.

27. The method of claim 22 wherein the first material and the expanding medium are the same materials.

28. The method of claim 22 wherein the second material is generally in the shape of a sphere.

29. The method of claim 22 wherein the second material is generally in the shape of a cylinder.

30. The method of claim 22 wherein the imaging device is within the expandable member.

31. The method of claim 22 wherein the imaging device is outside of the expandable member.

32. The method of claim 22 wherein the imaging device is an ultrasonic imaging head for ultrasound imaging.

33. The method of claim 22 wherein the imaging device is an optical fiber for optical coherent imaging.

34. The method of claim 22 further comprising performing at least one assessment of the lumen to determine at least one physical property of the lumen.

35. A method for assessing the shape and orientation of a lumen comprising:
deploying an expandable member to the location of a lumen within the body of a patient, the expandable member being attached to a catheter at or near a distal end thereof, the catheter having a collar located on the catheter shaft, the collar being a hollow member containing a liquid and gas bubbles to indicate the orientation of the device within a lumen.
expanding the expandable member to engage at least a portion of the lumen; and
imaging by an imaging device the gas bubbles in the collar to indicate the relative orientation of the expandable device with respect to the patient.

36. A medical device comprising:
a longitudinal body for inserting within a lumen or hollow portion of a patient; and
gravitational orientation means to indicate the orientation of the medical device within the lumen or hollow portion of the organ.
